(19) United States
(12) Patent Application Publication
(10) Pub. No.: US 2012/0078024 A1
(43) Pub. Date: Mar. 29, 2012

(54) Removal of Hydrogen From Dehydrogenation Processes

(75) Inventors: James R. Butler, League City, TX (US); James N. Waguespack, Spring, TX (US); Jason Clark, Houston, TX (US)

(73) Assignee: FINA TECHNOLOGY INC., Houston, TX (US)

(21) Appl. No.: 13/220,806

(22) Filed: Aug. 30, 2011

Related U.S. Application Data
(60) Provisional application No. 61/386,073, filed on Sep. 24, 2010.

(51) Int. Cl. C07C 5/32; B01J 19/00

(52) U.S. Cl. 585/312; 585/310; 585/319; 422/187

(57) ABSTRACT

A process and system for dehydrogenating certain hydrocarbons is disclosed. The process includes contacting a dehydrogenatable hydrocarbon with steam in the presence of a dehydrogenation catalyst to form hydrogen and a dehydrogenated hydrocarbon. Some of the hydrogen is then removed and some of the remaining dehydrogenatable hydrocarbon is dehydrogenated.

### FIELD

Embodiments generally relate to dehydrogenation processes. In particular, this disclosure relates to selective removal of hydrogen from dehydrogenation processes.

### BACKGROUND

Dehydrogenation is a chemical reaction that involves the removal of hydrogen from a compound. Dehydrogentation processes may be used to form various unsaturated organic compounds. For instance, some common dehydrogenation processes are ethane to ethylene, propane to propylene, butane to butylene or butadiene, as examples. Various vinyl compounds can be prepared by the catalytic dehydrogenation of corresponding alkyl compounds. Such reactions include the catalytic dehydrogenation of monoalkyl or polyalkyl aromatics, such as ethylene and diethylbenzene or the dehydrogenation of alkyl substituted polynuclear aromatic compounds, such as ethylnaphthalene. Perhaps the mostly widely used dehydrogenation process involves the dehydrogenation of ethylbenzene for the production of styrene.

Dehydrogenation is an equilibrium reaction. Commercial processes can be limited by the presence of the products, such as hydrogen. A need exists to improve dehydrogenation process efficiency.

### SUMMARY

Embodiments of the present disclosure include processes and systems for dehydrogenation.

In one embodiment of the present disclosure, a dehydrogenation process is disclosed. The dehydrogenation process includes providing a dehydrogenatable hydrocarbon and contacting the dehydrogenatable hydrocarbon with steam in the presence of a dehydrogenation catalyst to form a first product stream. The first product stream includes a first dehydrogenated hydrocarbon and hydrogen. The dehydrogenation process further includes passing the first product stream through a separation system adapted to remove hydrogen therefrom and form a second product stream and contacting the second product stream with steam in the presence of a dehydrogenation catalyst to form a third product stream. The third product stream includes a second dehydrogenated hydrocarbon and hydrogen, wherein the first and second dehydrogenated hydrocarbons may be the same hydrocarbon.

In another embodiment of the present disclosure, a dehydrogenation system is disclosed. The dehydrogenation system includes a first dehydrogenation reactor and an inorganic membrane. The membrane is adapted to separate hydrogen and is fluidically coupled to the first dehydrogenation reactor. The dehydrogenation system further includes a second dehydrogenation reactor fluidically coupled to the membrane.

In still another embodiment of the present disclosure a dehydrogenation process for converting ethyl benzene to styrene is disclosed. The process includes a first stream containing a first quantity of hydrogen and a second stream containing a second quantity of hydrogen different from the first quantity of hydrogen, wherein both first and second streams are separated by a membrane.

### BRIEF DESCRIPTION OF DRAWINGS

FIG. 1 illustrates an embodiment of a dehydrogenation process.

### DETAILED DESCRIPTION

### Introduction and Definitions

A detailed description will now be provided. Each of the appended claims defines a separate invention, which for infringement purposes is recognized as including equivalents to the various elements or limitations specified in the claims. The disclosure below is not limited to the embodiments, versions or examples described, which are included to enable a person having ordinary skill in the art to make and use the disclosed subject matter when the information in this patent is combined with available information and technology.

Various terms as used herein are shown below. To the extent a term used in a claim is not defined below, it should be given the broadest definition skilled persons in the pertinent art have given that term as reflected in printed publications and issued patents at the time of filing. Further, unless otherwise specified, all compounds described herein may be substituted or unsubstituted and the listing of compounds includes derivatives thereof.

Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations. For example, if the detailed description recites a range of from 1 to 5, that range includes all iterative ranges within that range including, for instance, 1.3-2.7 or 4.9-4.95.

Embodiments of the disclosure generally include dehydrogenation processes. Dehydrogenation processes generally include contacting a reactant, such as a C2 to C4 alkane or an alkyl aromatic hydrocarbon with a dehydrogenation catalyst to form a C2 to C4 alkene, or a vinyl aromatic hydrocarbon within a reaction vessel. The disclosure below is described with respect to alkyl aromatic compounds. It is understood by one skilled in the art with the benefit of this disclosure that the principles will apply likewise to other dehydrogenatable compounds, including, but not limited to, alkanes and parrafins. An alkyl aromatic hydrocarbon may include any alkyl aromatic hydrocarbon known to one skilled in the art, such as ethylbenzene, isopropylbenzene or ethyltoluene, for example.

As one of skill in the art will recognize with the benefit of this disclosure, the disclosure below is not limited with respect to the dehydrogenation catalyst. In certain embodiments, the dehydrogenation catalyst may include a reducible oxide of iron or vanadium, for example. As used herein, the term “reducible oxide” refers to an oxide which is reduced by contact with hydrocarbons when operating under dehydrogenation conditions.

The dehydrogenation catalyst may optionally be bound to, supported on or extruded with any suitable support material. The support material may include oxides of metals, such as titanium, zirconium, zinc, magnesium, thorium, silica, calcium, barium and aluminum, clays and zeolitic materials, such as metallo-silicates or metallo-alumino-phosphates (e.g., alumino-silicates, borosilicates, silico-alumino-phosphates), for example. In one specific, non-limiting, embodiment, the dehydrogenation catalyst includes a reducible vanadium oxide on a magnesium oxide support.

The dehydrogenation catalyst may further include one or more promoters, such as alkali or alkaline-earth metals, for example.

In one or more embodiments, the dehydrogenation catalyst may include as non-limiting examples metal oxides, such as CuO, ZnO—CuO, ZnO—CuO—Al₂O₃; CuCr₂O₃; ZnCr₂O₃, ZnO—CuO—Cr₂O₃, or metals, such as Ru, Rh, Ni, Co, Pd or Pt supported on a substrate such as silica or titania, for example.

The dehydrogenation catalyst may be prepared by methods known to one skilled in the art, such as absorption, precipitation, impregnation or combinations thereof, for example. See, U.S. Pat. No. 5,510,553, which is fully incorporated by reference herein.

The vinyl aromatic hydrocarbon formed in an embodiment of the present disclosure is generally dependent upon the alkyl aromatic hydrocarbon and may include styrene, α-methyl styrene or vinyl toluene, for example. The vinyl aromatic hydrocarbon may further be used for any suitable purpose and/or may undergo further processing, such as separation, for example.

In certain embodiments, the dehydrogenation processes are high temperature processes. As used herein, the term “high temperature” refers to process operation temperatures, such as reaction vessel and/or process line temperatures of from about 150° C. to about 1000° C., or from about 300° C. to about 800° C., or from about 500° C. to about 700° C. or from about 550° C. to about 650° C., for example.

The alkyl aromatic hydrocarbon may contact the dehydrogenation catalyst in the presence of an inert diluent, such as steam. Such contact may occur in any manner known to one skilled in the art. For example, the diluent may be added to the alkyl aromatic hydrocarbon prior to contact with the catalyst, for example. Although the amount of diluent contacting the alkyl aromatic hydrocarbon is determined by individual process parameters, the diluent may contact the alkyl aromatic hydrocarbon in a weight ratio of from about 0.01:1 to about 15:1, or from about 0.3:1 to about 10:1, or from about 0.6:1 to about 3:1, for example.

Embodiments of reactors that can be used with the present disclosure can include, by non-limiting examples: fixed bed reactors; fluid bed reactors; falling bed reactors and entrained bed reactors. Reactors capable of the elevated temperature and pressure as described herein, and capable of enabling contact of the reactants with the catalyst, can be considered within the scope of the present disclosure. Embodiments of the particular reactor system may be determined based on the particular design conditions and throughput, as by one of ordinary skill in the art, and are not meant to be limiting on the scope of the present disclosure. The dehydrogenation reactor may be of various configurations including a radial flow reactor such as disclosed in U.S. Pat. No. 5,358,698 or a linear or tubular reactor such as disclosed in U.S. Pat. Nos. 4,287,375 and 4,549,032, all of which are incorporated by reference herein.

It is contemplated that the dehydrogenation process may include a single or a plurality of stages. When utilizing a plurality of stages, such stages may be housed in a single reaction vessel, or in multiple reaction vessels, for example. In one or more embodiments, the multiple reaction vessels include series connected dehydrogenation reactions.

The product yields of dehydrogenation reactions is limited by equilibrium. The presence of hydrogen (often at significant levels) in the reaction vessel often requires significant inert diluents/steam feed rates in order to overcome equilibrium constraints. For example, the dehydrogenation processes may include a steam to alkyl aromatic hydrocarbon molar feed rate of from 6 to 15, for example. However, selective hydrogen removal from the dehydrogenation process may lower the level of steam required to overcome equilibrium constraints.

In one or more embodiments, series-connected dehydrogenation reactors generally include a separation system disposed between such adapted to remove hydrogen therefrom.

In other embodiments, the dehydrogenation system includes at least one in-situ separation system. As used herein, the term “in-situ” refers to disposal of the separation system within at least one reaction vessel.

In one or more embodiments, the separation system generally includes a membrane. The membrane is adapted to selectively remove hydrogen from the separation system without removal of steam and other products and/or reactants, for example. For example, the membrane may be adapted to remove at least 50% of the hydrogen introduced into the separation system. In one or more embodiments, the membrane is adapted to remove less than 10% of the steam introduced into the separation system.

It is contemplated herein that the term “membrane” may include the use of a single membrane or multiple membranes, depending on the required hydrogen migration or other process conditions, for example. The membrane generally includes a hydrogen permeable membrane. The hydrogen permeable membrane may be formed of any material which exhibits substantial permeability to hydrogen while being substantially impermeable to the larger molecules involved in the dehydrogenation reaction, such as the inert diluent, the alkyl aromatic hydrocarbon and the vinyl aromatic hydrocarbon, for example.

In one or more embodiments, the membrane is an inorganic membrane.

In one or more embodiments, the membrane is porous.

In one or more embodiments, the membrane is formed of a sintered metal, such as palladium, copper, alloys thereof and combinations thereof, for example. In one or more specific embodiments, the membrane is formed of palladium and from about 35 wt. % to about 45 wt. % copper, for example.

In one or more embodiments, the membrane is formed of a ceramic material, for example.

In one or more embodiments, the membrane may have a pore diameter of from about 0.5 nm to about 20,000 nm or less than 1 nm, for example.

In one or more embodiments, the membrane may have a thickness of about 2 μm or less, for example.

One or more embodiments include in-situ hydrogen separation via disposal of the membrane within the dehydrogenation reactor. For example, in one or more embodiments, the membrane is employed in the wall structure of the reactor. In yet another embodiment, the membrane is employed as a layer disposed within the reactor.

One or more embodiments include a separation unit disposed between various stages of the dehydrogenation reactor. Such separation unit may be disposed within the reactor or between multiple reactors, for example. When disposed between stages, the separation system may operate at a pressure of from about 2 psia to about 20 psia, for example. When disposed between stages, the separation system may operate at a temperature of from about 300° C. to about 700° C., for example.

The embodiments described herein result in the ability to overcome equilibrium constraints in dehydrogenation processes at lower temperatures, lower steam feed rates or combinations thereof without the requirement of condensation to remove the hydrogen formed therein.

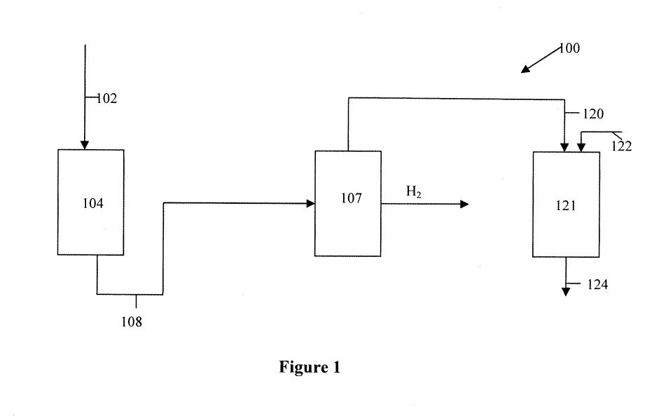

FIG. 1 illustrates a schematic block diagram of a specific, non-limiting, embodiment of a dehydrogenation process 100. Although not shown herein, the process stream flow may be modified based on unit optimization by those of skill in the art with the benefit of this disclosure. For example, additional process equipment, such as heat exchangers or separators, may be employed throughout the processes described herein and such placement is generally known to one skilled in the art. Further, while described below in terms of primary components, the streams indicated below may include any additional components as known to one skilled in the art.

The process 100 generally includes supplying an input stream 102 to a dehydrogenation system 104. The dehydrogenation system 104 is generally adapted to contact the input stream 102 with a dehydrogenation catalyst to form an output stream 108.

The input stream 102 generally includes the alkyl aromatic hydrocarbon and the output stream 108 generally includes the vinyl aromatic hydrocarbon. In addition, the input stream 102 may further include the inert diluent, for example.

The dehydrogenation system 104 generally includes one or more reaction zones, which are contained within one or more reaction vessels. In one embodiment, the reaction vessel generally includes a downflow reaction vessel. As used herein, downflow reaction vessels generally include circulating catalyst therethrough in a downward direction (versus upflow reactors) for contact with a feedstock and recovering the catalyst for regeneration and/or disposal.

Although illustrated as a single reaction zone, it is known to one skilled in the art with the benefit of this disclosure, that the reaction vessel may include one or a plurality or reaction zones, each having catalyst passing therethrough. Further, each reaction zone may be contained within a single reaction vessel or a plurality of reaction vessels, for example.

To aid in the flow of the applicable materials, such as steam, input and catalyst, the dehydrogenation system 104 generally utilizes a pressure drop. The applicable materials generally have a short residence time in the reaction zone, further aiding in maintaining the oxidation state in the desired tolerances. For example, the input may have a residence time of from about 0.5 milliseconds to about 30 seconds or from about 1 millisecond to about 15 seconds and the catalyst may have a residence time of from about 0.5 milliseconds to about 5 minutes or from about 1 millisecond to about 1 minute.

In the specific embodiment illustrated in FIG. 1, the output stream 108 is passed through a separation system 107 to selectively remove hydrogen therefrom forming a purified output stream 120. In one or more embodiments, the purified output stream 120 is passed to an additional dehydrogenation system 121 to form product stream 124.

While the foregoing is directed to certain embodiments, other and further embodiments may be devised without departing from the basic scope thereof and the scope thereof is determined by the claims that follow.

What is claimed is:

1. A dehydrogenation process comprising:
providing a dehydrogenatable hydrocarbon;
contacting the dehydrogenatable hydrocarbon with steam in the presence of a dehydrogenation catalyst to form a first product stream comprising a first dehydrogenated hydrocarbon and hydrogen;
passing the first product stream through a separation system adapted to remove hydrogen therefrom and form a second product stream; and
contacting the second product stream with steam in the presence of a dehydrogenation catalyst to form a third product stream comprising a second dehydrogenated hydrocarbon and hydrogen, wherein the first and second dehydrogenated hydrocarbons may be the same hydrocarbon.

2. The dehydrogenation process of claim 1, wherein the dehydrogenatable hydrocarbon is an alkane, or alkyl aromatic compound.

3. The dehydrogenation process of claim 2, wherein the dehydrogenatable hydrocarbon is an alkyl aromatic compound selected from the group consisting of ethylbenzene, isopropylbenzene and ethyl toluene.

4. The dehydrogenation process of claim 3, wherein the first and second dehydrogenated hydrocarbon is selected from the group consisting of styrene, α-methyl styrene, and vinyl toluene.

5. The dehydrogenation process of claim 1, wherein the separation system is a hydrogen-permeable membrane.

6. The dehydrogenation process of claim 5, wherein the hydrogen-permeable membrane is an inorganic membrane.

7. The dehydrogenation process of claim 6, wherein the inorganic membrane comprises sintered metal.

8. The dehydrogenation process of claim 7, wherein the sintered metal comprises palladium, copper, alloys thereof, or combinations thereof.

9. The dehydrogenation process of claim 8, wherein the sintered metal is comprised of a palladium/copper alloy, wherein the copper comprises about 35 wt % to about 45 wt % of the alloy.

10. The dehydrogenation process of claim 6, wherein the inorganic membrane comprises ceramic.

11. The dehydrogenation process of claim 6, wherein the hydrogen-permeable membrane includes pores having a diameter of less than 1 nanometer.

12. The dehydrogenation process of claim 6, wherein the hydrogen-permeable membrane has a thickness of 2 μm or less.

13. The dehydrogenation process of claim 6, wherein the hydrogen permeable membrane is adapted to remove 50% of the hydrogen in the first product stream.

14. The dehydrogenation process of claim 1, wherein the step of passing the first product stream through a separation system occurs within a dehydrogenation reactor.

15. The dehydrogenation process of claim 1, wherein the step of passing the first product stream through a separation system occurs outside a dehydrogenation reactor.

16. A dehydrogenation system comprising:
a first dehydrogenation reactor;
an inorganic membrane, the membrane adapted to separate hydrogen, the membrane fluidically coupled to the first dehydrogenation reactor; and
a second dehydrogenation reactor, wherein the second dehydrogenation reactor is fluidically coupled to the membrane.

17. The dehydrogenation system of claim 16, wherein the inorganic membrane is within the first dehydrogenation reactor.

18. The dehydrogenation system of claim 16, wherein the inorganic membrane comprises sintered metal or ceramic.

19. The dehydrogenation system of claim 18, wherein the inorganic membrane comprises sintered metal and the sintered metal comprises palladium, copper, alloys thereof, or combinations thereof.

20. The dehydrogenation system of claim 19, wherein the sintered metal is comprised of a palladium/copper alloy, wherein the copper comprises about 35 wt % to about 45 wt % of the alloy.

21. The dehydrogenation system of claim 18, wherein the hydrogen-permeable membrane includes pores having a diameter of less than 1 nanometer.

22. The dehydrogenation system of claim 18, wherein the hydrogen-permeable membrane has a thickness of 2 μm or less.

23. A dehydrogenation process for converting ethyl benzene to styrene comprising:
a first stream containing a first quantity of hydrogen and a second stream containing a second quantity of hydrogen different from the first quantity of hydrogen, wherein both first and second streams are separated by a membrane.
